(19) United States
(12) Patent Application Publication
(10) Pub. No.: US 2010/0124571 A1
(43) Pub. Date: May 20, 2010

(54) Use of pH-sensitive, Acid-Stable Metal-Binding Nanoparticles

(75) Inventors: Jinhyuk Fred Chung, Yusong-Ku (KR); Seyl Kim, Yusong-Ku (KR)

Correspondence Address: FENWICK & WEST LLP, SILICON VALLEY CENTER, 801 CALIFORNIA STREET, MOUNTAIN VIEW, CA, 94041, US

(73) Assignee: NANOMEDICINE RESEARCH CO., LTD., Yusong-Ku (KR)

(21) Appl. No.: 12/274,831

(22) Filed: Nov. 20, 2008

(51) Int. Cl. A61K 9/14; A61K 31/7004; A61K 31/4415; A61K 31/714

(52) U.S. Cl. 424/489; 514/23; 424/94.1; 514/27

(57) ABSTRACT

Methods of preventing and/or treating cancer are disclosed. The methods comprise administering to the patient a pharmaceutically effective amount of a water-soluble, acid-stable organometallic nanoparticles, optionally in combination with another therapeutic agent. In particular, nanoparticles that consist of polymerized citric acid and various different types of metals including, but not limited to, iron, calcium, zinc, silver and magnesium. These nanoparticles are acid-stability and self-degradation leading to constituent metal release when pH rises closer to the neutral pH of 7 or higher.

### FIELD OF INVENTION

The present invention relates to organometallic nanoparticles as target deliver system, more particularly, the invention relates to nanoparticles comprising chelating organic polymer and biologically active metals.

### BACKGROUND

Nanoparticles, having a typical diameter in the range of 1 to 1000 nm, have been used as catalysts, photocatalysts, adsorbents, and sensors. Recently, nanoparticles have been used for the treatment of diseases. Nanoparticles can bind or be linked to natural or synthetic substances such as drugs, medicaments, diagnostic agents, antisense oligonucleotides, proteins, plasmids etc. and carry such substances to target organs in the human or animal body, such as the brain, liver, kidneys and other organs (WO 95/22963 and WO 98/56361).

In particular, nanoparticles have been used for the treatment of cancers. Nanoparticles conjugated to drugs can be delivered to specific sites by either active targeting or by size-dependant passive targeting (Cancer Res. 1986; 46:6387-6392; J. Control. Release 1999; 62:253-262).

Active drug targeting is a method of selectively delivering anticancer elements to cancer cells by conjugating nanoparticles containing anticancer agents to recognition groups that bind or react with cancer cells. Nanoparticles-based drugs designed in this manner allow for controlled local release of drugs at specific drug targets defined by the recognition groups. Prime examples of active targeting method are lectin and carbohydrate, ligand and acceptor, or antibody and antigen (Farhan J. Ahmad, et al., Nanotechnology: A Revolution in the Making, The Pharma Review December 2005).

Lectin and carbohydrate binding is one of the conventional methods for specific drug delivery system. Lectin is a nonimmune protein that can recognize and bind with glycoprotein on the surfaces of cells. Interaction between lectin and a specified carbohydrate is achieved very specifically. Therefore, a carbohydrate moiety is used to bind the drug delivery system to lectin (direct lectin targeting), and the lectin can be used again as a targeting moiety to bind with a carbohydrate on the surface of a targeting cell (reverse lectin targeting).

Passive drug targeting, on the other hand, employs enhanced permeation and retention (EPR) effect to specifically target cancer cells. The EPR effect is a phenomenon which appears widely only in cancer cells and in angiogenic vascular structures of cancer. The EPR effect in cancer cells is characterized by non-selective absorption, permeation, and retention of macromolecules having a macromolecule size between 10 to 100 nm.

The passive targeting strategy has advantages over the active targeting strategy. First advantage is that passive designs do not require recognition groups, making production procedures and drug administration relatively simple. The second advantage is that the lack of specificity allows for general application of the passively designed anti-cancer drugs to any cancer types displaying EPR effect. Lastly, the consequent ability of such drugs to accumulate in cancer that come in contact with blood supply makes it easier to combat highly metastatic or mutating cancer.

Recently, passive targeting strategy has been employed against cancer cells by conjugating nanoparticles with conventional anti-cancer toxins such as radioactive/toxic heavy metals (Gadolinium, Holmium-166, Copper) or cytotoxins (FU-5)(H. Tokumitsu, et al., Chitosan-gadopentetic acid complex nanoparticles for gadolinium neutron capture therapy of cancer: preparation by novel emulsion droplet coalescence technique and characterization, Pharm. Res. (1999) 16: 1830-1835; Kim J K, et al., Long-term clinical outcome of phase IIb clinical trial of percutaneous injection with holmium-166/chitosan complexes (Milicam) for the treatment of small hepatocellular carcinoma Clin Cancer Res. (2006) 12(2): 543-8; Qi L, et al., Cytotoxic activities of chitosan nanoparticle and copper-loaded nanoparticles, Bioorg Med Chem. Lett. (2005) 15(5): 1397-9). These studies showed selective effectiveness of the conjugated toxins against cancers through enhanced necrosis, growth inhibition, and reduced metastasis when compared to those that were not fused with nanoparticles, as well as a reduction in side effects. However, the conjugated nanoparticles were shown to accumulate in brain/spinal cord/bone marrow (K. Ringe, et al. Nanoparticle Drug Delivery to the Brain, Encyclopedia of Nanotechnology (2004) volume 7: pages 91-104), resulting in critical toxicity and side effects in the respective organs that result from slow release of the toxic conjugates.

Therefore, there is a need for the development of anticancer drugs having wide and effective anticancer effect without short or long-term toxicity in order to effectively combat cancer. The present invention meets the need, and discloses water-soluble organometallic nanoparticles with biologically active metals with little or no toxicity.

### SUMMARY

The present invention provides compositions, dosage forms and methods for the treatment or prevention of diseases, such as cancer.

In one aspect, the invention provides methods for treating or preventing cancer in a patient which comprises administering to the patient water-soluble, acid-stable nanoparticles wherein the nanoparticle comprises an organic compound of formula I

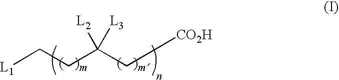

wherein L₁, L₂, and L₃ are independently selected to be H, OH, halogen, NR₁R₂, SH, SO₃R₃, or CO₂R₄, wherein R₁, R₂, R₃, and R₄ can independently be H or lower alkyl, and m, m′, and n can be independently selected to be an integer between 0 and 20 and a metal and/or a metal salt wherein the nanoparticle has a size between about 1 nm to about 500 nm.

Specific examples of cancers that can be treated by this method include, but are not limited to, leukemias, angiogenic disorders, cancer of the head, neck, eye, mouth, throat, esophagus, chest, bone, lung, colon, rectum, stomach, prostate, breast, ovaries, kidney, liver, pancreas, and brain.

In another aspect of the invention, the invention provides methods for treating or preventing cancer in a patient which comprises administering to the patient water-soluble, acid-stable nanoparticles and another therapeutic agent. The additional therapeutic agent can be pyrrolidine dithiocarbamate (PDTC), vitamin C, vitamin B12, vitamin B6, folate, dichloroacetate (DCA), 2-deoxyglucose (2 DG), interferon receptor agonist, an anti-cancer agent, an anti-angiogenic agent, and combinations thereof. Preferably, the additional therapeutic agent is selected such that the combination of the nanoparticles and the therapeutic agent have a synergistic effect.

These and other aspects of the present invention will become evident upon reference to the following detailed description. In addition, various references are set forth herein which describe in more detail certain procedures or compositions, and are therefore incorporated by reference in their entirety.

### BRIEF DESCRIPTION OF DRAWINGS

FIG. 1 illustrates the effect of nanoparticles containing iron (OFe) on H460 human lung cancer and B16F10 mouse melanoma cell lines. FIG. 1A illustrates the effect of different concentrations of OFe on H460. FIG. 1B illustrates the effect of different concentrations of OFe on B16F10.

FIG. 2 illustrates the effect of nanoparticles containing iron and calcium (OFeCa) on H460 human lung cancer and B16F10 mouse melanoma cell lines. FIG. 2A illustrates the effect of different concentrations of OFeCa on H460. FIG. 2B illustrates the effect of different concentrations of OFeCa on B16F10.

FIG. 3 illustrates the effect of nanoparticles containing iron and zinc (OFeZn) on H460 human lung cancer and B16F10 mouse melanoma cell lines. FIG. 3A illustrates the effect of different concentrations of OFeZn on H460. FIG. 3B illustrates the effect of different concentrations of OFeZn on B16F10.

FIG. 4 illustrates the effect of nanoparticles containing iron, calcium and zinc (OFeCaZn) on H460 human lung cancer and B16F10 mouse melanoma cell lines. FIG. 4A illustrates the effect of different concentrations of OFeZn on H460. FIG. 4B illustrates the effect of different concentrations of OFeZn on B16F10.

FIG. 5 illustrates the effect of nanoparticles containing iron and silver (OFeAg) on H460 human lung cancer cell line.

FIG. 6 illustrates the effect of nanoparticles containing at least iron, calcium, magnesium and zinc (OFeCa-1) on H460 human lung cancer.

### DETAILED DESCRIPTION

### I. Definitions

Unless otherwise stated, the following terms used in this application, including the specification and claims, have the definitions given below. It must be noted that, as used in the specification and the appended claims, the singular forms “a,” “an” and “the” include plural referents unless the context clearly dictates otherwise. Definition of standard chemistry terms may be found in reference works, including Carey and Sundberg (1992) “Advanced Organic Chemistry 3^{rd} Ed.” Vols. A and B, Plenum Press, New York. The practice of the present invention will employ, unless otherwise indicated, conventional methods of mass spectroscopy, protein chemistry, biochemistry, recombinant DNA techniques and pharmacology, within the skill of the art.

The terms “effective amount” or “pharmaceutically effective amount” refer to a nontoxic but sufficient amount of the agent to provide the desired biological result. That result can be reduction and/or alleviation of the signs, symptoms, or causes of a disease, or any other desired alteration of a biological system. An appropriate “effective” amount in any individual case may be determined by one of ordinary skill in the art using routine experimentation.

As used herein, the terms “treat” or “treatment” are used interchangeably and are meant to indicate a postponement of a disease, such as a cancer, and/or a reduction in the severity of the disease that will or are expected to develop. The terms further include ameliorating existing cancer disease symptoms, preventing additional symptoms, and ameliorating or preventing the underlying metabolic causes of symptoms.

By “pharmaceutically acceptable” or “pharmacologically acceptable” is meant a material which is not biologically or otherwise undesirable, i.e., the material may be administered to an individual without causing any undesirable biological effects or interacting in a deleterious manner with any of the components of the composition in which it is contained.

By “physiological pH” or a “pH in the physiologically acceptable range” or “near neutral pH” is meant a pH in the range of approximately 6.0 to 8.0 inclusive, more typically in the range of approximately 6.5 to 7.6 inclusive.

As used herein, the term “subject” encompasses mammals and non-mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. The term does not denote a particular age or gender.

The term “pharmaceutically acceptable salt” of a compound means a salt that is pharmaceutically acceptable and that possesses the desired pharmacological activity of the parent compound. Such salts, for example, include:

(1) acid addition salts, formed with inorganic acids such as hydrochloric acid, hydrobromic acid, sulfuric acid, nitric acid, phosphoric acid, and the like; or (2) salts formed when an acidic proton present in the parent compound either is replaced by a metal ion, e.g., an alkali metal ion, an alkaline earth ion, or an aluminum ion; or coordinates with an organic base. It should be understood that a reference to a pharmaceutically acceptable salt includes the solvent addition forms or crystal forms thereof, particularly solvates or polymorphs. Solvates contain either stoichiometric or non-stoichiometric amounts of a solvent, and are often formed during the process of crystallization. Hydrates are formed when the solvent is water, or alcoholates are formed when the solvent is alcohol.

The term “optional” or “optionally” means that the subsequently described event or circumstance may or may not occur, and that the description includes instances where the event or circumstance occurs and instances where it does not. For example, the phrase “optionally another drug” means that the patient may or may not be given a drug other than the organometallic nanoparticles. “Another drug” as used herein is meant any chemical material or compound suitable for administration to a mammalian, preferably human, which induces a desired local or systemic effect. In general, this includes: anorexics; anti-infectives such as antibiotics and antiviral agents, including many penicillins and cephalosporins; analgesics and analgesic combinations; antiarrhythmics; antiarthritics; antiasthmatic agents; anticholinergics; anticonvulsants; antidiabetic agents; antidiarrheals; antihelminthics; antihistamines; antiinflammatory agents; antimigraine preparations; antinauseants; antineoplastics; antiparkinsonism drugs; antipruritics; antipsychotics; antipyretics; antisense agents; antispasmodics; cardiovascular preparations including calcium channel blockers and beta-blockers such as pindolol; antihypertensives; central nervous system stimulants; cough and cold preparations, including decongestants; diuretics; gastrointestinal drugs, including H₂-receptor antagonists; sympathomimetics; hormones such as estradiol and other steroids, including corticosteroids; hypnotics; immunosuppressives; muscle relaxants; parasympatholytics; psychostimulants; sedatives; tranquilizers; thrombolytics; neuroprotectants; radical scavengers and vasodilators.

### II. Overview

The present invention discloses compositions of nanoparticles and the use of the nanoparticles for the treatment or prevention of cancer. The nanoparticles of the invention comprise pH-sensitive, self-dissociating organometallic nanoparticles. The nanoparticles are stable under acidic conditions and dissociate near neutral pH or under physiological conditions. The nanoparticles of the invention have a dissociation rate such that majority of the dissociate occurs in the cancer cells and not in the circulatory system, and the dissociation in the cells releases metal in high enough concentration to kill the cancer cell. The metal of the nanoparticle can be selected to have cytotoxic properties, either alone or in combination with other drugs, but not toxic to the normal cells at the therapeutic concentrations.

Thus, the nanoparticles of the invention are water-soluble organometallic nanoparticles comprising an organic molecule that can form an ester and that can bind metal and/or metal ions, and a metal. The nanoparticles can be used for the treatment or prevention of a cancer. Without being bound to a theory, the nanoparticles of the invention act as anticancer agents via enhanced permeation and retention (EPR) effect.

Nanoparticles of the present invention can be retained selectively in cancer cells via EPR effect. And upon sufficient accumulation, the nanoparticles give direct/indirect oxidative stress to the cancer cells, causing specific toxicity at site. The nanoparticles of the present invention having such anticancer mechanism consist of a multidentate metal chelating organic polymer and a plurality of minerals, some of which are capable of generating direct oxidative stress to the cancer cells, and others with biological functionality to trigger biological reactions/cycles/cascades.

### III. Nanoparticles

The nanoparticles for use in the present invention can be synthesized using the methods described in the co-pending and co-owned application entitled “Synthesis of pH-Sensitive, Acid-Stable, Metal-Binding Nanoparticles Using Citric Acid and Metal, and Their Anticancer Applications Thereof.” Typically, the nanoparticles comprise an organic molecule that can form an ester and that can bind or chelate metal and/or metal ions, and a metal and/or a metal ion. Thus, the organic molecule can have a structure of formula I:

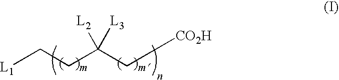

wherein L₁, L₂, and L₃ can be independently selected to be H, OH, halogen, NR₁R₂, SH, SO₃R₃, and CO₂R₄, wherein R₁, R₂, R₃, and R₄ can independently be H or lower alkyl, and m, m′, and n can be independently selected to be an integer between 0 and 20. Preferably, the organic acid is a biological compound, such as citric acid.

The metal and/or metal ion in the nanoparticles can be selected such that the metal has low toxicity to normal cells at the therapeutically effective does, can be easily removed/detoxified from a body, and can directly causing oxidation in cells. Thus, the metal can be selected to be include iron, magnesium, manganese, titanium, cesium, silver, gold, platinum, nickel, or combinations thereof, and can also include minerals such as calcium, zinc, potassium or sodium that have no inherent toxicity but are capable of causing indirect biological stress by triggering biological reactions/cycles/cascades in any cell upon significant accumulation and steady release.

The nanoparticles can be prepared by incubating the organic compound with the metal or the metal salt. The reaction mixture can be allowed to stir until the reaction is complete. Typical reaction times range from about 20 minutes to about 2 months, depending on the desired nanoparticle. The reaction mixture can be stirred at a temperature of about −20° C. to about 100° C., more preferably about 0° C. to about 70° C., even more preferably about 20° C. to about 50° C., and any temperature in between.

The water-soluble organometallic nanoparticles thus produces can have an average particle size of about 0.1 nm to about 600 nm, more preferably about 1.0 nm to about 30 nm and most preferably about 2 nm to 20 nm. The nanoparticles can thus have a particle size of 0.1, 1, 2, 3, 4, 5, 6, 7, 8, 9, or 10 nm, and up to about 500 nm. In another aspect, the nanoparticles can have a range of particle size, or diameter distribution. For example, the nanoparticles can have particle sizes in the range of about 5 nm and about 20 nm in size, about 6 nm and about 25 nm in size, or about 5 nm and about 500 nm in size.

### IV. Treatment Methods

The present invention provides methods for treating a disorder in an individual where the disorder is amenable to treatment using acid-stable metal-binding nanoparticles including methods for treating a proliferative disorder, such as cancer, angiogenesis-mediated disorders, and fibrotic disorders.

In one aspect, the present invention provides methods of treating or preventing cancer in a patient. The methods generally involve administering to a patient in need thereof an amount of a therapeutically effective amount of the acid-stable, metal-binding nanoparticles. The methods can optionally comprise analysis of a biological sample from the patient with a pre-treatment biological sample from the patient for cell proliferation, and adjusting the dose of the nanoparticles based on the results of the comparison. In some embodiments, the methods further involve administering an effective amount of at least a second therapeutic agent, such as, for example, pyrrolidine dithiocarbamate (PDTC), vitamin C, vitamin B12, vitamin B6, folate, dichloroacetate (DCA), 2-deoxyglucose (2 DG), interferon receptor agonist, an anti-cancer agent, or combinations thereof.

The methods of the invention are useful for treating a wide variety of cancers, including carcinomas, sarcomas, leukemias, and lymphomas.

Carcinomas that can be treated using the methods of the invention include, but are not limited to, esophageal carcinoma, hepatocellular carcinoma, basal cell carcinoma (a form of skin cancer), squamous cell carcinoma (various tissues), bladder carcinoma, including transitional cell carcinoma (a malignant neoplasm of the bladder), bronchogenic carcinoma, colon carcinoma, colorectal carcinoma, gastric carcinoma, lung carcinoma, including small cell carcinoma and non-small cell carcinoma of the lung, adrenocortical carcinoma, thyroid carcinoma, pancreatic carcinoma, breast carcinoma, ovarian carcinoma, prostate carcinoma, adenocarcinoma, sweat gland carcinoma, sebaceous gland carcinoma, papillary carcinoma, papillary adenocarcinoma, cystadenocarcinoma, medullary carcinoma, renal cell carcinoma, ductal carcinoma in situ or bile duct carcinoma, choriocarcinoma, seminoma, embryonal carcinoma, Wilm's tumor, cervical carcinoma, uterine carcinoma, testicular carcinoma, osteogenic carcinoma, epithelial carcinoma, and nasopharyngeal carcinoma, etc.

Sarcomas that can be treated using a method of the invention include, but are not limited to, fibrosarcoma, myxosarcoma, liposarcoma, chondrosarcoma, chordoma, osteogenic sarcoma, osteosarcoma, angiosarcoma, endotheliosarcoma, lymphangiosarcoma, lymphangioendotheliosarcoma, synovioma, mesothelioma, Ewing's sarcoma, leiomyosarcoma, rhabdomyosarcoma, and other soft tissue sarcomas.

Other solid tumors that can be treated include, but are not limited to, glioma, astrocytoma, medulloblastoma, craniopharyngioma, ependymoma, pinealoma, hemangioblastoma, acoustic neuroma, oligodendroglioma, menangioma, melanoma, neuroblastoma, and retinoblastoma.

Leukemias that can be treated using a subject method include, but are not limited to, a) chronic myeloproliferative syndromes (neoplastic disorders of multipotential hematopoietic stem cells); b) acute myelogenous leukemias (neoplastic transformation of a multipotential hematopoietic stem cell or a hematopoietic cell of restricted lineage potential; c) chronic lymphocytic leukemias (CLL; clonal proliferation of immunologically immature and functionally incompetent small lymphocytes), including B-cell CLL, T-cell CLL prolymphocytic leukemia, and hairy cell leukemia; and d) acute lymphoblastic leukemias (characterized by accumulation of lymphoblasts). Lymphomas that can be treated using a subject method include, but are not limited to, B-cell lymphomas (e.g., Burkitt's lymphoma); Hodgkin's lymphoma; and the like.

In another aspect, the methods of the invention are useful for treating angiogenic disorders, such as any disease characterized by pathological neovascularization. Such disorders include, but are not limited to, solid tumors, hemangiomas, rheumatoid arthritis, atherosclerosis, fibrotic disorders, including idiopathic pulmonary fibrosis (IPF), liver fibrosis, and renal fibrosis; but also include BPH, vascular restenosis, arteriovenous malformations (AVM), retinopathies, including diabetic retinopathy, meningioma, hemangiomas, thyroid hyperplasias (including Grave's disease), neovascular glaucoma, neovascularization associated with corneal injury, neovascularization associated with corneal transplantation, neovascularization associated with corneal graft, psoriasis, angiofibroma, hemophilic joints, hypertrophic scars, osler-weber syndrome, age-related macular degeneration, pyogenic granuloma retrolental fibroplasia, scleroderma, trachoma, vascular adhesions, synovitis, dermatitis, an inflammatory bowel disease such as, for example, Crohn's disease or ulcerative colitis, and endometriosis.

In one aspect of the invention, the methods involve treating or preventing the occurrence of cancer in a patient by administering the nanoparticles of the invention, optionally in combination with at least a second therapeutic agent. Suitable additional therapeutic agents include, but are not limited to, pyrrolidine dithiocarbamate (PDTC), vitamin C, vitamin B12, vitamin B6, folate including physiological equivalents of folic acid such as pharmaceutically acceptable salts thereof, 5-methyltetrahydrofolate and polyglutamate forms thereof as occur naturally, interferon receptor agonist, an anti-cancer agent (e.g., an anti-neoplastic agent, an anti-proliferative agent, a cytotoxic agent), an anti-angiogenic agent, an anti-inflammatory agent, an anti-fibrotic agent, a hematopoietic agent, a TNF-α antagonist, or combinations thereof.

Additional therapeutic agents also include compounds which disrupt microtubule function, such as estramustine, epothilone, curacin-A, colchicine, methotrexate, and paclitaxel, vinblastine, vincristine, and 4-tert-butyl-[3-(2-chloroethyl)ureido]benzene (tBCEU).

Antineoplastic agents are well known and include, for example, the following agents and their congeners or analogues; camptothecin and its analogues such as topotecan and irinotecan, altretamine, aminoglutethimide, azathioprine, cyclosporine, dacarbazine, dactinomycin, daunorubicin, docetaxel, doxorubicin, gemcitabine, etoposide, hydroxyurea, irinotecan, interferon, methylmelamines, mitotane, paclitaxel and analogues such as docetaxel, procarbazine HCl, teniposide, topotecan, vinblastine sulfate, vincristine sulfate, and vinorelbine. Other antineoplastic agents further include antibiotics and their congeners and analogues such as actinomycin, bleomycin sulfate, idarubicin, plicamycin, mitomycin C, pentostatin, and mitoxantrone; antimetabolites such as cytarabine, fludarabine, fluorouracil, floxuridine, cladribine, methotrexate, mercaptopurine, and thioguanine; alkylating agents such as busulfan, carboplatin, cisplatin, and thiotepa; nitrogen mustards such as melphalan, cyclophosphamide, ifosfamide, chlorambucil, and mechlorethamine; nitrosureas such as carmustine, lomustine, and streptozocin; and toxins such as ricin.

In another aspect of the invention, the subject methods comprise administering to an individual in need thereof an effective amount of the nanoparticles and an effective amount of an interferon receptor agonist. The interferon receptor agonist can be, for example, INFERGEN™, PEGylated IFN-α, PEGASYS™, PEG-INTRON™, and the like.

The methods are effective to reduce a tumor load by at least about 5%, at least about 10%, at least about 20%, at least about 25%, at least about 50%, at least about 75%, at least about 85%, or at least about 90%, up to total eradication of the tumor, when compared to a suitable control. Thus, therapeutically effective amounts of nanoparticles and/or a second therapeutic agent are amounts that, in monotherapy or combination therapy, are sufficient to reduce tumor load by at least about 5%, at least about 10%, at least about 20%, at least about 25%, at least about 50%, at least about 75%, at least about 85%, or at least about 90%, up to total eradication of the tumor, when compared to a suitable control. In an experimental animal system, a suitable control may be the tumor load present in a genetically identical animal lot treated with nanoparticles monotherapy or combination therapy. In non-experimental systems, a suitable control may be the tumor load present before administering the nanoparticles monotherapy or combination therapy. Other suitable controls can be a placebo control.

Whether a tumor load has been decreased can be determined using any known method, including, but not limited to, measuring solid tumor mass; counting the number of tumor cells using cytological assays; fluorescence-activated cell sorting (e.g., using antibody specific for a tumor-associated antigen) to determine the number of cells bearing a given tumor antigen; computed tomography scanning, magnetic resonance imaging, and/or x-ray imaging of the tumor to estimate and/or monitor tumor size; measuring the amount of tumor-associated antigen in a biological sample, e.g., blood; and the like.

In another aspect of the invention, the methods of the invention are effective to reduce the growth rate of a tumor by at least about 5%, at least about 10%, at least about 20%, at least about 25%, at least about 50%, at least about 75%, at least about 85%, or at least about 90%, up to total inhibition of growth of the tumor, when compared to a suitable control. Thus, in these embodiments, effective amounts of a nanoparticles, optionally in combination with a second therapeutic agent are amounts that, in monotherapy or combination therapy, are sufficient to reduce tumor growth rate by at least about 5%, at least about 10%, at least about 20%, at least about 25%, at least about 50%, at least about 75%, at least about 85%, or at least about 90%, up to total inhibition of tumor growth, when compared to a suitable control. In an experimental animal system, a suitable control may be the growth rate of a tumor in a genetically identical animal not treated with nanoparticles monotherapy or combination therapy. In non-experimental systems, a suitable control can be the growth rate of a tumor observed before administering the nanoparticle monotherapy or combination therapy. Other suitable controls can be a placebo control.

Whether growth of a tumor is inhibited can be determined using any known method, including, but not limited to, an in vitro proliferation assay such as a ³H-thymidine uptake assay, and the like.

### V. Synergy

Further, the methods described above can be used to identify compounds that exhibit synergy as measured by the synergism index. The synergism index (SI) can be calculated by any of the art known equations. Typically, the values are based on the amount needed to achieve a specified end point. The end point selected for these studies can be a 10% reduction in the tumor load. Synergy Index (SI)=(QA/Qa)+(QB/Qb) where: QA=quantity of nanoparticle A in mixture producing the end point; Qa=quantity of nanoparticle A, acting alone, producing the end point; QB=quantity of compound B in mixture producing the end point; Qb=quantity of compound B, acting alone, producing the end point. If SI is less than 1, synergism exists; if SI is greater than 1, antagonism exists; if SI is equal to 1, an additive effect exists. Thus, synergism is defined as a combination index of less than one.

The compositions of the invention, such as compositions comprising acid-stable, metal-binding nanoparticles, have a superior effect as a medicine. The compositions of the present invention have low toxicity and causes no or few side effects. Therefore, the compositions are useful as, for example, a therapeutic agent for the prophylaxis or treatment of various diseases, such as primary cancer, metastasis or recurrence of malignant tumor (e.g., prostate cancer).

While the compositions of the present invention has a superior effect even when used solely, the effect can be further promoted by using the compound in combination with other pharmaceutical preparations and therapies. Examples of the preparation and therapy to be combined include, but not limited to, pyrrolidine dithiocarbamate (PDTC), vitamin C, vitamin B12, vitamin B6, folate including include physiological equivalents of folic acid such as pharmaceutically acceptable salts thereof, 5-methyltetrahydrofolate and polyglutamate forms thereof as occur naturally, interferon receptor agonist, an anti-cancer agent (e.g., an anti-neoplastic agent, an anti-proliferative agent, a cytotoxic agent), an anti-angiogenic agent, an anti-inflammatory agent, an anti-fibrotic agent, a hematopoietic agent, a TNF-α antagonist, or combinations thereof. Preferably, the combination has a synergistic index of less than 1, preferably less than about 0.9, more preferably less than about 0.8.

### VI. Pharmaceutical Formulations and Modes of Administration

The methods described herein use pharmaceutical compositions comprising the acid-stable, metal-binding nanoparticles described above, either alone or in combination with another compound with which it has synergistic effect, together with one or more pharmaceutically acceptable excipients or vehicles, and optionally other therapeutic and/or prophylactic ingredients. Such excipients include liquids such as water, saline, glycerol, polyethyleneglycol, hyaluronic acid, ethanol, etc. Suitable excipients for non-liquid formulations are also known to those of skill in the art. Pharmaceutically acceptable salts can be used in the compositions of the present invention and include, for example, mineral acid salts such as hydrochlorides, hydrobromides, phosphates, sulfates, and the like; and the salts of organic acids such as acetates, propionates, malonates, benzoates, and the like. A thorough discussion of pharmaceutically acceptable excipients and salts is available in Remington's Pharmaceutical Sciences, 18th Edition (Easton, Pa.: Mack Publishing Company, 1990).

Additionally, auxiliary substances, such as wetting or emulsifying agents, biological buffering substances, surfactants, and the like, may be present in such vehicles. A biological buffer can be virtually any solution which is pharmacologically acceptable and which provides the formulation with the desired pH, i.e., a pH in the physiologically acceptable range. Examples of buffer solutions include saline, phosphate buffered saline, Tris buffered saline, Hank's buffered saline, and the like.

Depending on the intended mode of administration, the pharmaceutical compositions may be in the form of solid, semi-solid or liquid dosage forms, such as, for example, tablets, suppositories, pills, capsules, powders, liquids, suspensions, creams, ointments, lotions or the like, preferably in unit dosage form suitable for single administration of a precise dosage. The compositions will include an effective amount of the selected drug in combination with a pharmaceutically acceptable carrier and, in addition, may include other pharmaceutical agents, adjuvants, diluents, buffers, etc.

The invention includes a pharmaceutical composition comprising a compound of the present invention including isomers, racemic or non-racemic mixtures of isomers, or pharmaceutically acceptable salts or solvates thereof together with one or more pharmaceutically acceptable carriers, and optionally other therapeutic and/or prophylactic ingredients.

In general, the compounds of this invention will be administered in a therapeutically effective amount by any of the accepted modes of administration. Suitable dosage ranges depend upon numerous factors such as the severity of the disease to be treated, the age and relative health of the subject, the potency of the compound used, the route and form of administration, the indication towards which the administration is directed, and the preferences and experience of the medical practitioner involved. One of ordinary skill in the art of treating such diseases will be able, without undue experimentation and in reliance upon personal knowledge and the disclosure of this application, to ascertain a therapeutically effective amount of the compounds of this invention for a given disease.

### VII. Dosage

The amount of acid-stable organometallic nanoparticles that is combined with one or more excipients to produce a single dosage form will necessarily vary depending upon the host treated and the particular route of administration.

Generally, a formulation intended for oral administration to humans will generally contain for example from 0.01 mg to 1000 mg of acid-stable organometallic nanoparticles per Kg of bodyweight combined with an appropriate and convenient amount of excipients.

Dosage unit forms will generally contain about 0.1 mg to about 500 mg of an acid-stable organometallic nanoparticles.

More specifically, a formulation comprising acid-stable organometallic nanoparticles, for example, intended for oral administration to humans will generally contain for example from 0.01 mg to 1 mg of acid-stable organometallic nanoparticles per Kg of bodyweight combined with an appropriate and convenient amount of excipients.

However, it will be readily understood that it may be necessary to vary the dose of the acid-stable organometallic nanoparticles administered in accordance with well known medical practice in order to take account of the nature and severity of the condition or disease under treatment, any concurrent therapy, and of the age, weight, genotype and sex of the patient receiving treatment.

Generally, in therapeutic use, it is envisaged that a composition according to the invention would be administered so that a dose of the acid-stable organometallic nanoparticles is received which is generally in the range 0.00002 to 100 mg/kg/day, or 0.001 to 5000 mg/day more specifically, 0.05-1000 mg/day and 0.1-500 mg/day or 0.01 to 80 mg of active agent per Kg of bodyweight daily given if necessary in divided doses.

More specifically, for a composition comprising the acid-stable organometallic nanoparticles, in therapeutic use, it is envisaged that a composition according to the invention would be administered so that a dose of the acid-stable organometallic nanoparticles is received which is generally in the range 00002 to 100 mg/kg/day, or 0.01 to 500 mg/day. More specifically, from between 0.05-1000 mg/day and 0.1-500 mg/day or 0.01 to 80 mg of the acid-stable organometallic nanoparticles per Kg of bodyweight daily given if necessary in divided doses. All ranges throughout this specification are inclusive. For example from 0.01 to 100 includes the values 0.01 and 100.

### EXAMPLES

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

### Example 1

### Effectiveness of OFe Against Cancer Cell Lines

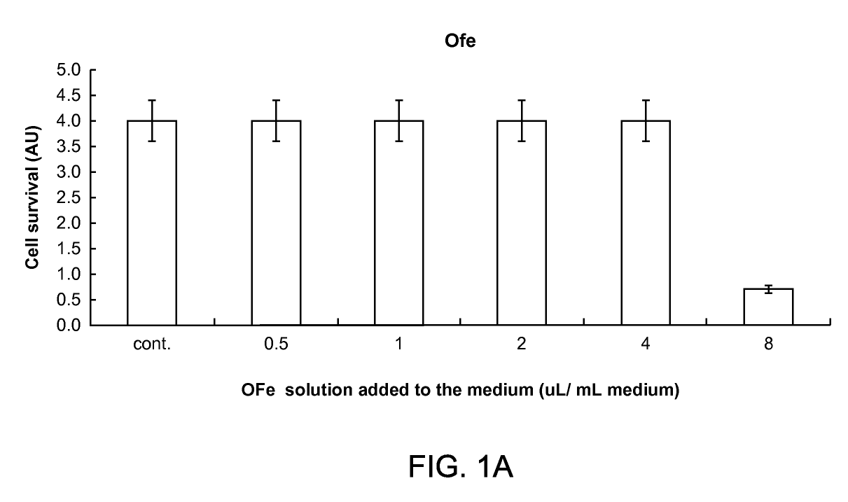

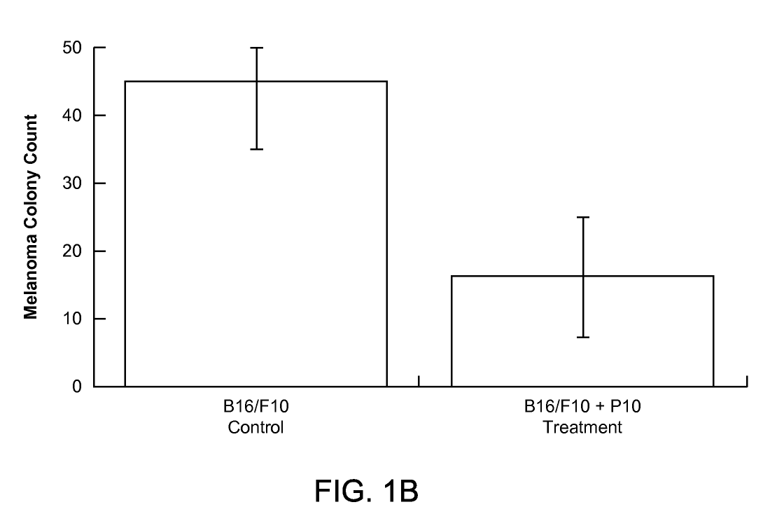

The organometallic nanoparticles (OFe) was prepared as described in the co-pending application and was tested in vitro against H460 human lung cancer and B16F10 mouse melanoma cell lines. The OFe organometallic nanoparticles contain only iron. An aqueous solution of OFe was prepared that had an iron concentration of 11,000 ppm. The cell lines were cultured on a dish in the presence or absence of OFe and tested using Cell Counting Kit-8™ (Dojindo, Tokyo). The results are illustrated in FIG. 1A for H460 and FIG. 1B for B16F10 cell lines. The data show OFe reduces the cell survival in both cell lines.

### Example 2

### Effectiveness of OFeCa Against Cancer Cell Lines

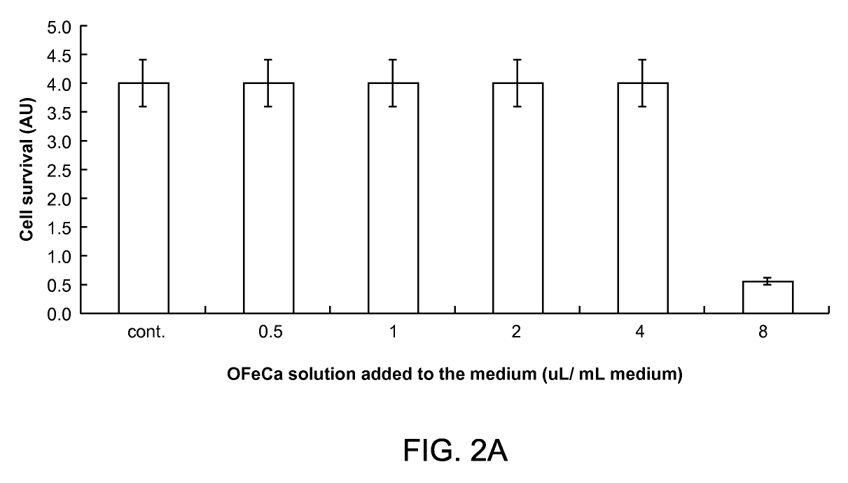

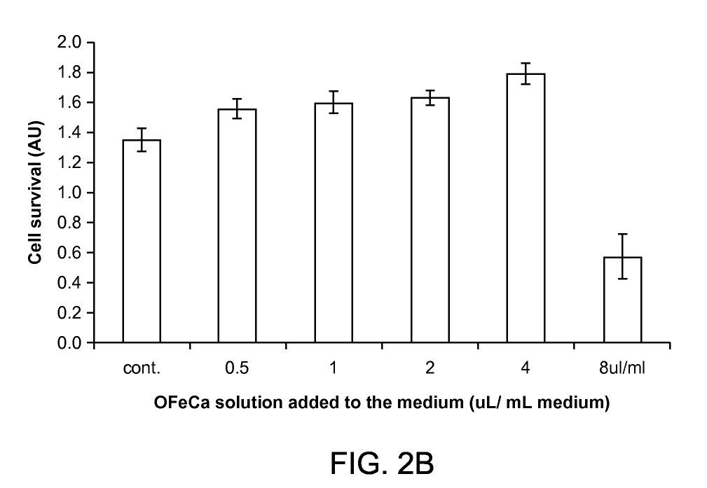

The organometallic nanoparticles (OFeCa) was prepared as described in the co-pending application and was tested in vitro against H460 human lung cancer and B16F10 mouse melanoma cell lines. The OFeCa organometallic nanoparticles contain iron and calcium. An aqueous solution of OFeCa was prepared that had an iron concentration of 11,000 ppm and calcium concentration of about 8,000 ppm. The cell lines were cultured on a dish in the presence or absence of OFeCa and tested using Cell Counting Kit-8™ (Dojindo, Tokyo). The results are illustrated in FIG. 2A for H460 and FIG. 2B for B16F10 cell lines. The data show OFeCa reduces the cell survival in both cell lines.

### Example 3

### Effectiveness of OFeZn Against Cancer Cell Lines

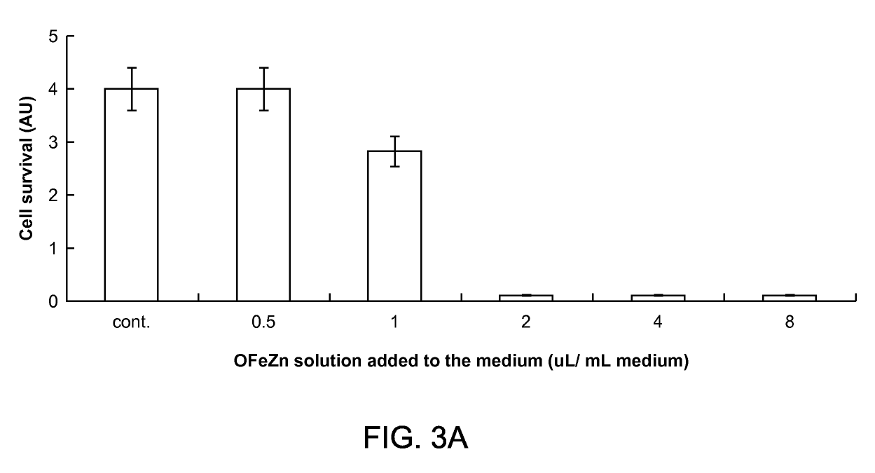

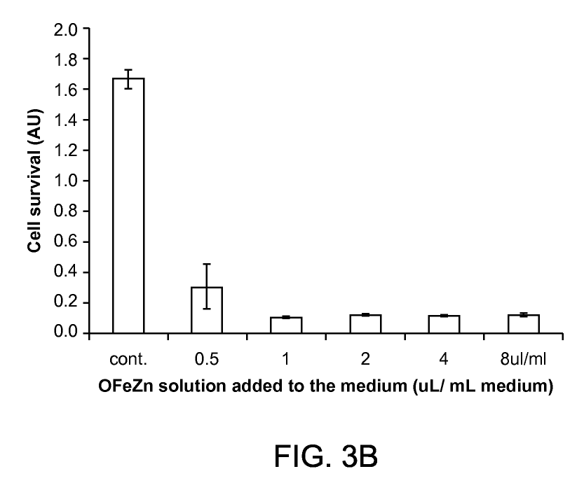

The organometallic nanoparticles (OFeZn) was prepared as described in the co-pending application and was tested in vitro against H460 human lung cancer and B16F10 mouse melanoma cell lines. The OFeZn organometallic nanoparticles contain iron and zinc. An aqueous solution of OFeZn was prepared that had an iron concentration of 11,000 ppm and zinc concentration of about 9,500 ppm. The cell lines were cultured on a dish in the presence or absence of OFeZn and tested using Cell Counting Kit-8™ (Dojindo, Tokyo). The results are illustrated in FIG. 3A for H460 and FIG. 3B for B16F10 cell lines. The data show OFeZn reduces the cell survival in both cell lines, and is effective at much lower concentrations of the nanoparticles, indicating OFeZn is more potent.

### Example 4

### Effectiveness of OFeCaZn Against Cancer Cell Lines

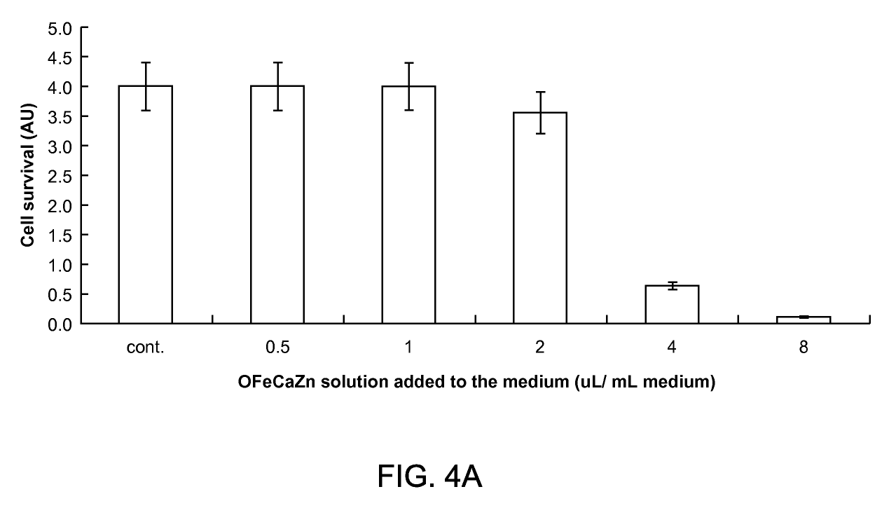

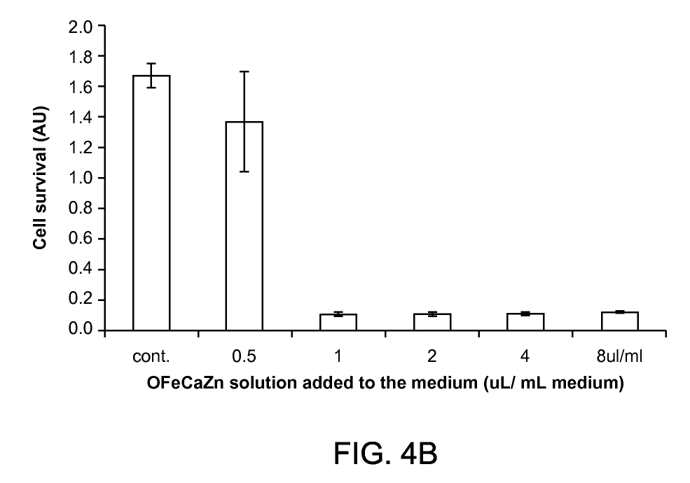

The organometallic nanoparticles (OFeCaZn) was prepared as described in the co-pending application and was tested in vitro against H460 human lung cancer and B16F10 mouse melanoma cell lines. The OFeCaZn organometallic nanoparticles contain iron, calcium, and zinc. An aqueous solution of OFeCaZn was prepared that had an iron concentration of 11,000 ppm, calcium concentration of about 8,000 ppm, and zinc concentration of about 9,500 ppm. The cell lines were cultured on a dish in the presence or absence of OFeCaZn and tested using Cell Counting Kit-8™ (Dojindo, Tokyo). The results are illustrated in FIG. 4A for H460 and FIG. 4B for B16F10 cell lines. The data show OFeCaZn reduces the cell survival in both cell lines.

### Example 5

### Effectiveness of OFeCaAg Against Cancer Cell Lines

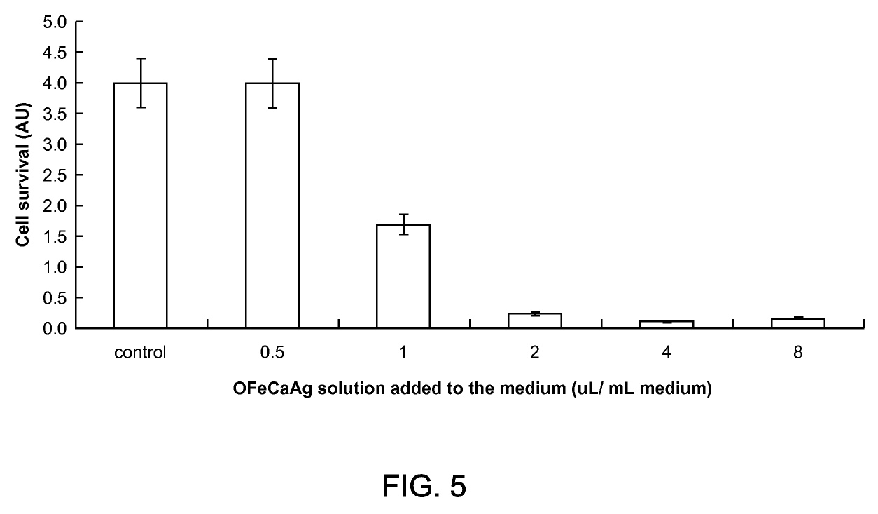

The organometallic nanoparticles (OFeCaAg) was prepared as described in the co-pending application and was tested in vitro against H460 human lung cancer cell line. The OFeCaAg organometallic nanoparticles contain iron, calcium and silver. An aqueous solution of OFeCaAg was prepared that had an iron concentration of 11,000 ppm, calcium concentration of about 8,000 ppm, and silver concentration of about 3,000 ppm. The H460 cell line was cultured on a dish in the presence or absence of OFeCaAg and tested using Cell Counting Kit-8™ (Dojindo, Tokyo). The results are illustrated in FIG. 5. The data show OFeCaAg reduces the cell survival in the cancer cell line is more effective than OFe or OFeCa.

### Example 6

### Effectiveness of OFeCa-1 Against Cancer Cell Lines

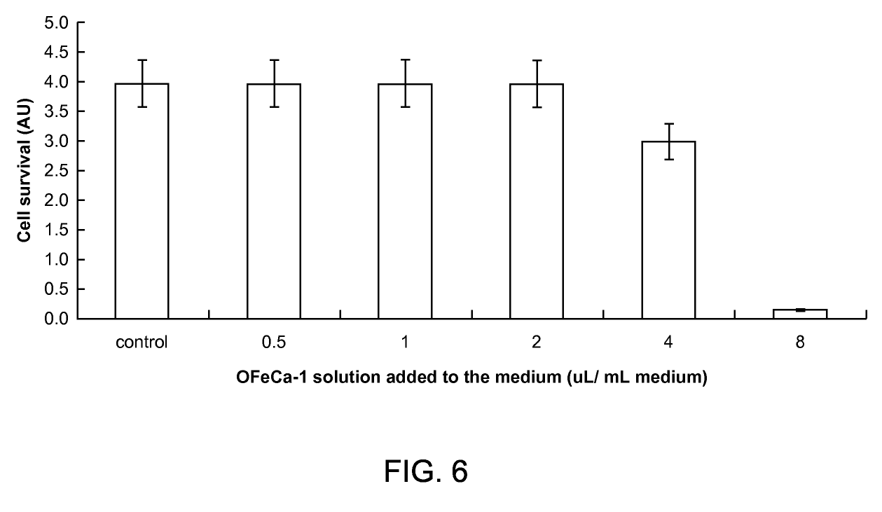

The organometallic nanoparticles (OFeCa-1) was prepared as described in the co-pending application and was tested in vitro against H460 human lung cancer cell line. The OFeCa-1 organometallic nanoparticles contain iron, calcium, zinc, magnesium and other metals. An aqueous solution of OFeCa-1 was prepared that had an iron concentration of 11,000 ppm, calcium concentration of about 3,400 ppm, and zinc concentration of about 500 ppm. The cell line was cultured on a dish in the presence or absence of OFeCa-1 and tested using Cell Counting Kit-8™ (Dojindo, Tokyo). The results are illustrated in FIG. 6. The data show OFeCa-1 reduces the cell survival at least as effectively as OFe or OCa.

While the invention has been particularly shown and described with reference to a preferred embodiment and various alternate embodiments, it will be understood by persons skilled in the relevant art that various changes in form and details can be made therein without departing from the spirit and scope of the invention. All printed patents and publications referred to in this application are hereby incorporated herein in their entirety by this reference.

1. A method treating or preventing cancer in a patient, the method comprising administering to the patient water-soluble, acid-stable nanoparticles wherein the nanoparticle comprises an organic compound of formula I 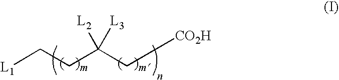
wherein L₁, L₂, and L₃ are independently selected to be H, OH, halogen, NR₁R₂, SH, SO₃R₃, or CO₂R₄, wherein R₁, R₂, R₃, and R₄ can independently be H or lower alkyl, and m, m′, and n can be independently selected to be an integer between 0 and 20 and a metal and/or a metal salt wherein the nanoparticle has a size between about 1 nm to about 500 nm.

2. The method of claim 1, wherein the nanoparticle dissociates near neutral pH.

3. The method of claim 1, wherein the organic compound can form an ester and can chelate a metal.

4. The method of claim 3, wherein the organic compound is citric acid, isocitric acid, glutamic acid, or 3-aminopentanedioic acid.

5. The method of claim 4, wherein the organic compound is citric acid.

6. The method of claim 1, wherein the metal is selected from the group consisting of Fe, Ca, Mg, Mn, K, Na, Zn, Ti, Si, Cs, Cu, Ag, Au, Pt, Ni, and combinations thereof.

7. The method of claim 6, wherein the metal is Fe, Ca, Zn, Ag, or combination thereof.

8. The method of claim 1, further comprising a therapeutic agent.

9. The method of claim 8, wherein the therapeutic agent is selected from the group consisting of pyrrolidine dithiocarbamate (PDTC), vitamin C, vitamin B12, vitamin B6, folate, dichloroacetate (DCA), 2-deoxyglucose (2 DG), interferon receptor agonist, an anti-cancer agent, an anti-angiogenic agent, and combinations thereof.

10. The method of claim 9, wherein therapeutic agent is vitamin B12.

11. The method of claim 9, wherein therapeutic agent is vitamin B6.

12. The method of claim 8, wherein the nanoparticles and the therapeutic agent are selected to result in a synergy index of less than 1.

13. The method of claim 12, wherein the synergy index is less than about 0.8.

14. The method of claim 1, wherein the subject is human.
